(19) United States
(12) Patent Application Publication
(10) Pub. No.: US 2024/0309470 A1
(43) Pub. Date: Sep. 19, 2024

(54) KOMPETITIVE ALLELE-SPECIFIC POLYMERASE CHAIN REACTION MOLECULAR MARKERS CLOSELY LINKED WITH COTTON FIBRE LENGTH QUANTITATIVE TRAIT LOCUS AND APPLICATIONS THEREOF

(71) Applicant: INSTITUTE OF COTTON RESEARCH OF CAAS, Anyang (CN)

(72) Inventors: Xiongming DU, Anyang (CN); Yingrui ZHAO, Anyang (CN); Shoupu HE, Anyang (CN); Baojun CHEN, Anyang (CN); Zhao'e PAN, Anyang (CN); Yinhua JIA, Anyang (CN); Baoyin PANG, Anyang (CN); Jingjing WANG, Anyang (CN)

(21) Appl. No.: 18/605,587

(22) Filed: Mar. 14, 2024

(30) Foreign Application Priority Data
Mar. 15, 2023 (CN) 202310247574.8

(51) Int. Cl. C12Q 1/6895

(52) U.S. Cl. CPC C12Q 1/6895; C12Q 1/68; C12Q 2600/13; C12Q 2600/16

(57) ABSTRACT

A combination of KASP molecular markers closely linked with cotton fibre length QTL and applications thereof are provided in the present disclosure, belonging to the technical field of cotton breeding. At least one KASP molecular marker combination described in the following (1)-(3) in included: (1) four KASP molecular markers closely linked with FL5 interval in cotton fibre length QTL; (2) four KASP molecular markers closely linked with FL3 interval in cotton fibre length QTL; and (3) four KASP molecular markers closely linked to PL2 interval in cotton fibre length QTL.

### CROSS-REFERENCE TO RELATED APPLICATIONS

This application claims priority to Chinese Patent Application NO. 202310247574.8. filed on Mar. 15, 2023, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)). identifying the names of each file, the date of creation of each file, and the size of each file in bytes as follows:
File name: sequence_347158PPH
Creation date: Mar. 11, 2024
Byte size: 33,565

### TECHNICAL FIELD

The present disclosure relates to the technical field of cotton breeding, and in particular to kompetitive allele-specific polymerase chain reaction (KASP) molecular markers closely linked with cotton fibre length quantitative trait locus (QTL) and applications thereof.

### BACKGROUND

Cotton is one of the most important cash crops in the world, and cotton fibre is an important raw material for the textile industry. At present, the cotton cultivars used mainly include Asian cotton, sea island cotton and upland cotton. Native to Central America, upland cotton is well-known for its adaptability, high yields, long fibres and other excellent qualities. The economic value of cotton lies mainly in its application to a wide range of woven fabrics, making it one of the world's dominant agricultural crops. To improve the economic value of cotton, it is important to improve the length of the cotton fibre.

The fibre length in cotton is a quantitative trait controlled by multiple genes, and quantitative trait locus (QTL) mapping is an effective technique for analysing the genetic basis of complex quantitative traits, including mainly two methods: linkage analysis and association analysis. Linkage analysis is mainly based on populations constructed from genetically diverse parents, with high mapping ability, but the two-parent populations lack genetic diversity and there is a large number of recombination events. Association analyses are mainly based on germplasm resource populations obtained through extensive collection of germplasm resources, and the populations are rich in genetic and phenotypic variation, enabling the detection of substantial QTL. There has been a large number of QTL related to fibre length located in cotton, but it is a question of how to apply these QTL of fibre length to precision molecular breeding in cotton.

QTL of fibre length possessed by the parents may be aggregated to recombinant monocultures using multiparental hybrid populations, and the recombinant monocultures that aggregate fibre length QTL may be used for breeding. The presence or absence of a recombinant monoculture with a single fibre length QTL may be identified and screened using molecular markers, as well as a monoculture that aggregates multiple fibre length QTLs.

### SUMMARY

The objective of the present disclosure is to provide kompetitive allele-specific polymerase chain reaction (KASP) molecular markers closely linked with cotton fibre length quantitative trait locus (QTL) and applications thereof, so as to solve the problems existing in the prior art. The KASP molecular marker of the present disclosure enables the polymeric identification of multiple QTL of fibre length, allowing a substantial increase in the fibre length of cotton varieties, and providing a strong guarantee for the screening of superior varieties of cotton plants.

In order to achieve the above objectives, the present disclosure provides the following scheme.

A combination of KASP molecular markers closely linked with cotton fibre length QTL, including a combination of KASP molecular markers as described in at least one of (1)-(3) below:

(1) KASP molecular markers KASP-G41, KASP-G42, KASP-G43 and KASP-G44 closely linked to FLS interval in cotton fibre length QTL;

(2) KASP molecular markers KASP-G21, KASP-G22, KASP-G23 and KASP-G24 closely linked to FL3 interval in cotton fibre length QTL; and

(3) KASP molecular markers KASP-G31, KASP-G32, KASP-G33 and KASP-G34 closely linked to FL2 interval in cotton fibre length QTL;

a physical location of a single-nucleotide polymorphism (SNP) site of the KASP-G41 is 88,396,617 bp on A07 chromosome, being a C/T mutation;

a physical location of an SNP site of the KASP-G42 is 88,437,416 bp on A07 chromosome, being an A/G mutation;

a physical location of an SNP site of the KASP-G43 is 88,459,489 bp on A07 chromosome, being a C/T mutation;

a physical location of an SNP site of the KASP-G44 is 88,469,638 bp on A07 chromosome, being a C/T mutation;

a physical location of an SNP site of the KASP-G21 is 61,894,952 bp on A09 chromosome, being a C/G mutation;

a physical location of an SNP site of the KASP-G22 is 61,890,95l bp on A09 chromosome, being a C/G mutation;

a physical location of an SNP site of the KASP-G23 is 61,976,868 bp on A09 chromosome, being a C/T mutation;

a physical location of an SNP site of the KASP-G24 is 62,027,231 bp on A09 chromosome, being a C/G mutation;

a physical location of an SNP site of the KASPG31 is 24,669,170 bp on D11 chromosome, being a G/T mutation;

a physical location of an SNP site of the KASP-G32 is 24,671,829 bp on D11 chromosome, being a C/G mutation;

a physical location of an SNP site of the KASP-G33 is 24,676,672 bp on D11 chromosome, being a C/T mutation; and

a physical location of an SNP site of the KASP-G34 is 24,567,513 bp on D11 chromosome, being an A/G mutation.

The present disclosure also provides a KASP primer pair combination closely linked with FL5 interval in cotton fibre length QTL, and the KASP primer pair combination includes primers as shown in the following table:

| S/N of SNP | KASP marker name | Primer type | S/N of sequence |
|---|---|---|---|
| SNP1 | KASP-G41 | X | SEQ ID NO. 1 |
| | | Y | SEQ ID NO. 2 |
| | | R | SEQ ID NO. 3 |
| SNP2 | KASP-G42 | X | SEQ ID NO. 4 |
| | | Y | SEQ ID NO. 5 |
| | | R | SEQ ID NO. 6 |
| SNP3 | KASP-G43 | X | SEQ ID NO. 7 |
| | | Y | SEQ ID NO. 8 |
| | | R | SEQ ID NO. 9 |
| SNP4 | KASP-G44 | X | SEQ ID NO. 10 |
| | | Y | SEQ ID NO. 11 |
| | | R | SEQ ID NO. 12 |

The present disclosure also provides a KASP primer pair combination closely linked with FL3 interval in cotton fibre length QTL, and the KASP primer pair combination includes primers as shown in the following table:

| S/N of SNP | KASP marker name | Primer type | S/N of sequence |
|---|---|---|---|
| SNP5 | KASP-G21 | X | SEQ ID NO. 13 |
| | | Y | SEQ ID NO. 14 |
| | | R | SEQ ID NO. 15 |
| | KASP-G22 | X | SEQ ID NO. 16 |
| SNP6 | | Y | SEQ ID NO. 17 |
| | | R | SEQ ID NO. 18 |
| | KASP-G23 | X | SEQ ID NO. 19 |
| SNP7 | | Y | SEQ ID NO. 20 |
| | | R | SEQ ID NO. 21 |
| | KASP-G24 | X | SEQ ID NO. 22 |
| SNP8 | | Y | SEQ ID NO. 23 |
| | | R | SEQ ID NO. 24 |

The present disclosure also provides a KASP primer pair combination closely linked with FL2 interval in cotton fibre length QTL, and the KASP primer pair combination includes primers shown in the following table:

| S/N of SNP | KASP marker name | Primer type | S/N of sequence |
|---|---|---|---|
| SNP9 | KASP-G31 | X | SEQ ID NO. 25 |
| | | Y | SEQ ID NO. 26 |
| | | R | SEQ ID NO. 27 |
| | KASP-G32 | X | SEQ ID NO. 28 |
| SNP10 | | Y | SEQ ID NO. 29 |
| | | R | SEQ ID NO. 30 |
| | KASP-G33 | X | SEQ ID NO. 31 |
| SNP11 | | Y | SEQ ID NO. 32 |
| | | R | SEQ ID NO. 33 |
| | KASP-G34 | X | SEQ ID NO. 34 |
| SNP12 | | Y | SEQ ID NO. 35 |
| | | R | SEQ ID NO. 36 |

The present disclosure also provides a method for identifying cotton fibre length traits by using a single cotton fibre length QTL molecular marker, including the following steps:

S1, conducting a KASP molecular marker genotyping detection on a cotton material to be tested by using the KASP primer pair combination;

S2, identifying the cotton material to be tested as a cotton plant with long fibre trait when an amplified signal as described in any of the following (1)-(3) is read:

(1) an amplified signal of primer type X respectively detected by the KASP-G41, the KASP-G42, the KASP-G43 and the KASP-G44 when the KASP primer pair combination is employed:

(2) an amplified signal of primer type Y respectively detected by the KASP-G21, the KASP-G22, the KASP-G23 and the KASP-G24 when the KASP primer pair combination is employed; and

(3) an amplified signal of primer type X respectively detected by the KASP-G31, the KASP-G32, the KASP-G33 and the KASP-G34 when the KASP primer pair combination is employed.

The present disclosure also provides a method for identifying cotton fibre length traits by using a plurality of cotton fibre length QTL molecular markers, including the following steps:

(1) carrying out KASP molecular marker genotyping detection on a cotton material to be tested by using the KASP primer pair combination;

(2) identifying the cotton material to be tested as a cotton plant with long fibre trait when an amplified signal shown below is read: an amplified signal with primer type X detected respectively by the KASP-G41, the KASP-G42, the KASP-G43 and the KASP-G44, an amplified signal with primer type Y detected respectively by the KASP-G21, the KASP-G22, the KASP-G23 and the KASP-G24, and an amplified signal with primer type X respectively detected by the KASP-G31, the KASP-G32, the KASP-G33 and the KASP-G34.

The present disclosure also provides a kit for identifying cotton fibre length traits, including at least one of the primer pair combinations.

The present disclosure also provides an application in preparing a kit for identifying cotton fibre length traits.

The present disclosure also provides an application in cotton breeding.

Optionally, the application is to identify cotton fibre length traits.

The present disclosure discloses the following technical effects.

The present disclosure is beneficial to molecular mark assisted selection breed of cotton fibre length and promotes the improvement of cotton fibre length.

Molecular marker-assisted screening of individual fibre length QTL using the KASP molecular markers of the present disclosure in the FC population (isolated population of 4133Bt/M-8124-1159//Lumianyan NO. 28/Jimian NO. 20///Dalingmian NO. 69/Zhongmiansuo 12/Zhongmiansuo 41/Uganda NO. 3 cross), YZ population (isolated population from the cross of Zhong AR40772/Luyuan 343/Suyuan 7235/Suyou 6070//Suyuan 04-3/Suyou 6003//Sumian 9108/J02-508) and FY population (4133Bt/M-8124-1159//Lumianyan NO. 28/Jimian 20//Dalingmian NO. 69/Zhongmiansuo 12//Zhongmiansuo 41/Uganda NO. 3///Zhong AR40772/Luyuan 343//Suyuan 7235/Suyou 6070//Suyuan 04-3/Suyou 6003//Sumian 9108/J02-508) may screen long fibre homozygous allelic materials and long fibre homozygous allelic materials, and the average fibre length olong fibre homozygous allelic materials is significantly longer than that of long fibre homozygous allelic materials by 0.5-1.4 mm. This indicates that the KASP molecular marker of the present disclosure may be used to screen for materials with longer fibres.

(3) The molecular polymerization screening of multiple fibre length QTLs is carried out on FC population, YZ population and FY population by using the KASP molecular markers of the present disclosure, so that materials with three fibre length QTLs are screened, and the average fibre length of long fibre homozygous allelic materials containing three fibre length QTLs is 3.1 mm longer than that of short fibre homozygous allelic materials containing three fibre length QTLs, suggesting that the KASP molecular markers may realize the polymerization identification of multiple fibre length QTLs, and may greatly increase the fibre length of cotton varieties.

### BRIEF DESCRIPTION OF THE DRAWINGS

In order to explain the embodiments of the present disclosure or the technical scheme in the prior art more clearly, the drawings needed in the embodiments are briefly introduced below. Obviously, the drawings described below are only some embodiments of the present disclosure, and other drawings may be obtained according to these drawings without creative work for ordinary people in the field.

FIG. 1A shows the frequency distribution of fibre length trait of F2 isolated population.

FIG. 1B shows the frequency distribution of fibre length trait of Fas family.

FIG. 2 shows the linkage disequilibrium structural domains of closely linked KASP molecular markers in the FL5 interval of the cotton fibre length QTL.

FIG. 3 shows the linkage disequilibrium structural domains of closely linked KASP molecular markers in the FL2 interval of the cotton fibre length QTL.

FIG. 4 shows the linkage disequilibrium structural domains of closely linked KASP molecular markers in the FL3 interval of the cotton fibre length QTL.

FIG. 5 is a process illustrating a method for identifying cotton fibre length traits by using a single cotton fibre length QTL molecular marker provided by the present disclosure.

### DETAILED DESCRIPTION OF THE EMBODIMENTS

A number of exemplary embodiments of the present disclosure are now described in detail, and this detailed description should not be considered as a limitation of the present disclosure, but should be understood as a more detailed description of certain aspects, characteristics and embodiments of the present disclosure.

It should be understood that the terminology described in the present disclosure is only for describing specific embodiments and is not used to limit the present disclosure. In addition, for the numerical range in the present disclosure, it should be understood that each intermediate value between the upper limit and the lower limit of the range is also specifically disclosed. Intermediate values within any stated value or stated range, as well as each smaller range between any other stated value or intermediate values within the stated range are also included in the present disclosure. The upper and lower limits of these smaller ranges can be independently included or excluded from the range.

Unless otherwise specified, all technical and scientific terms used herein have the same meaning as commonly understood by one of ordinary skill in the art to which this present disclosure relates. Although the present disclosure only describes the preferred methods and materials, any methods and materials similar or equivalent to those described herein can also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes can be made to the specific embodiments of the present disclosure without departing from the scope or spirit of the present disclosure. Other embodiments will be apparent to the skilled person from the description of the present disclosure. The description and example of that present disclosure are exemplary only.

The terms “including”, “comprising”, “having” and “containing” used in this specification are all open terms, which means including but not limited to.

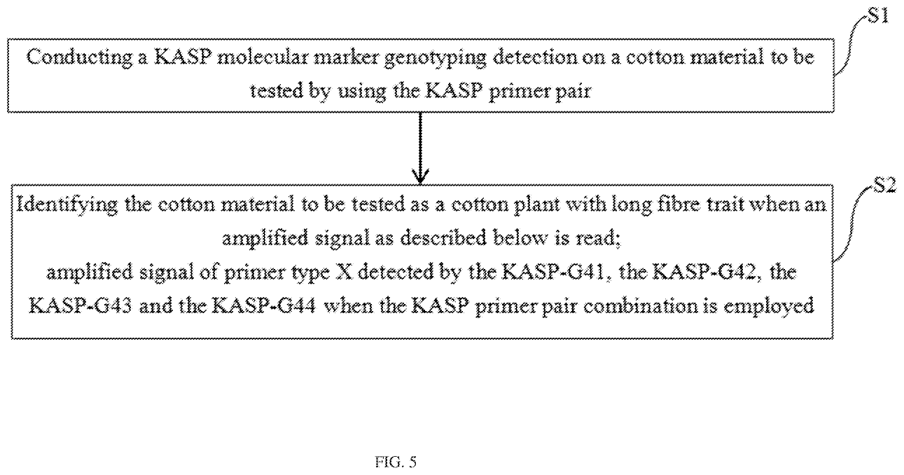

The present disclosure provides a method for identifying cotton fibre length traits by using a single cotton fibre length QTL molecular marker, including the following steps as shown in FIG. 5:

S1, conducting a KASP molecular marker genotyping detection on a cotton material to be tested by using the KASP primer pair; and

S2, identifying the cotton material to be tested as a cotton plant with long fibre trait when an amplified signal as described below is read:

amplified signal of primer type X detected by the KASP-G41, the KASP-G42, the KASP-G43 and the KASP-G44 when the KASP primer pair combination is employed.

Description of terms:

the FL5 referred to in the present disclosure is located on the A07 chromosome, with a physical location of 88,391,473 bp to 88,557,465 bp;

the FL3 referred to in the present disclosure is located on chromosome A09, and its physical location is 61,835,562 bp to 62,123,785 bp; and

the FL2 referred to in the present disclosure is located on chromosome D11, and its physical location is 24,509,312 bp to 24,800,082 bp.

### EMBODIMENT 1

The development and application of KASP molecular markers closely linked with cotton fibre length QTL specifically include the following steps.

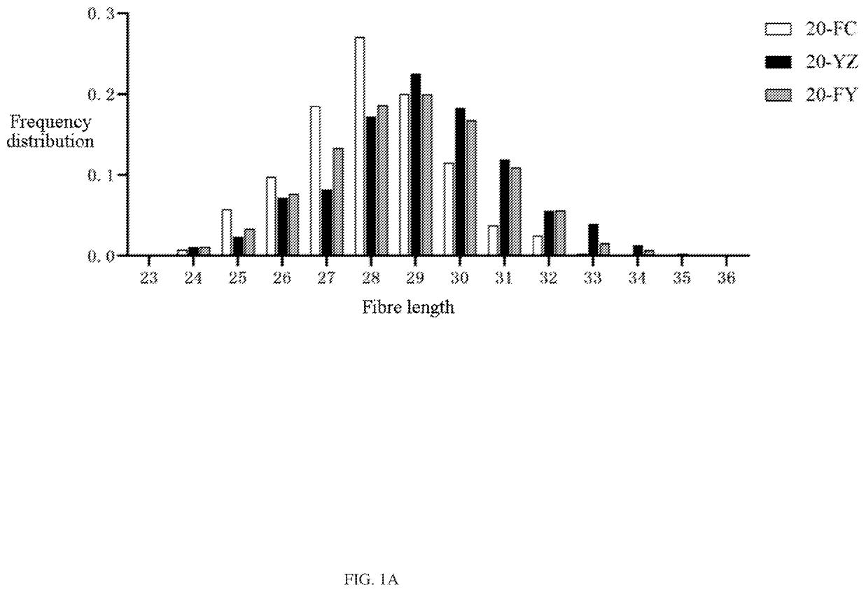

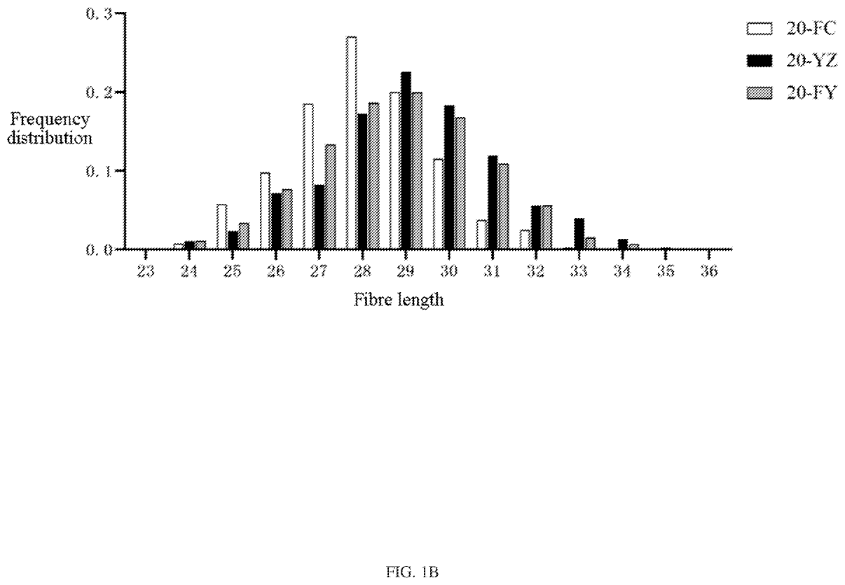

(1) The isolated population of the cross referred to as FC population (4133Bt/M-8124-1159//Lumianyan NO. 28/Jimian20//Dalingmian NO. 69/ZHongmiansuo 12/Zhongmiansuo 41/Uganda NO. 3), the isolated population of the cross referred to as YZ population (Zhong AR40772/Luyuan 343/Suyuan 7235/Suyou 6070//Suyuan 04-3/Suyou 6003//Sumian 9108/J02-508) and the isolated population of the cross referred to as FY population (4133Bt/M-8124-1159//Lumianyan NO. 28/Jimian 20//Dalingmian NO. 69/Zhongmiansuo 12//Zhongmiansuo 41/Uganda NO. 3///Zhong AR40772/Luyuan 343//Suyuan 7235/Suyou 6070///Suyuan 04-3/Suyou 6003//Sumian 9108/J02-508) are constructed, and the F: isolated population and the F2:3 family are tested for fibre length with results as shown in FIG. 1A and FIG. 1B.

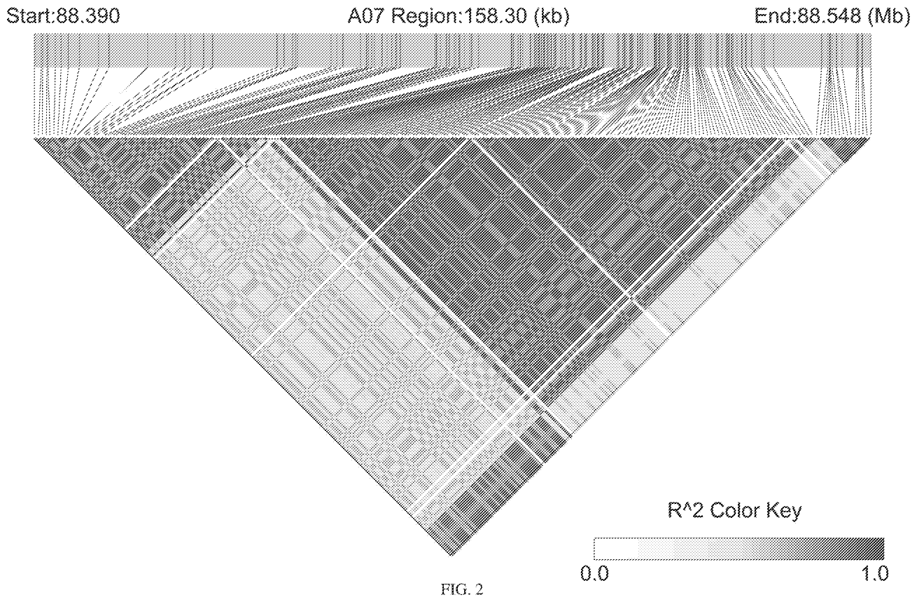

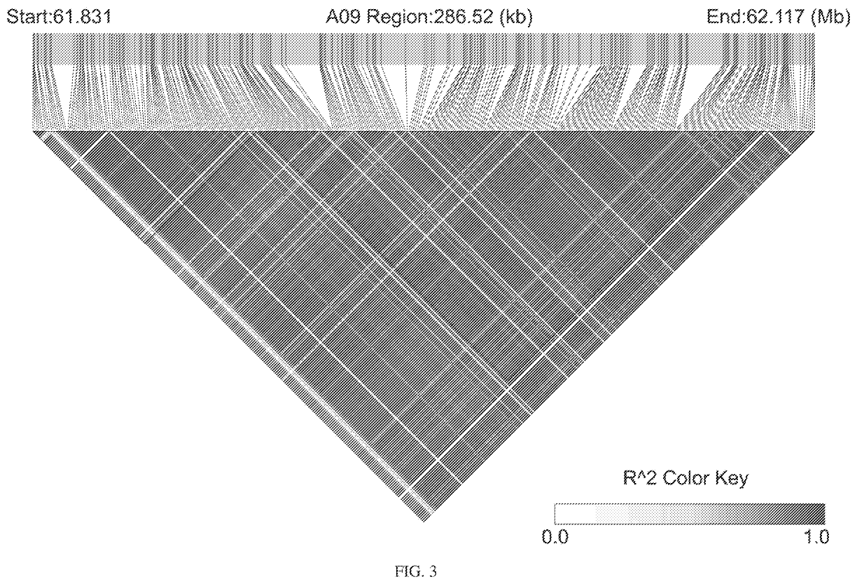

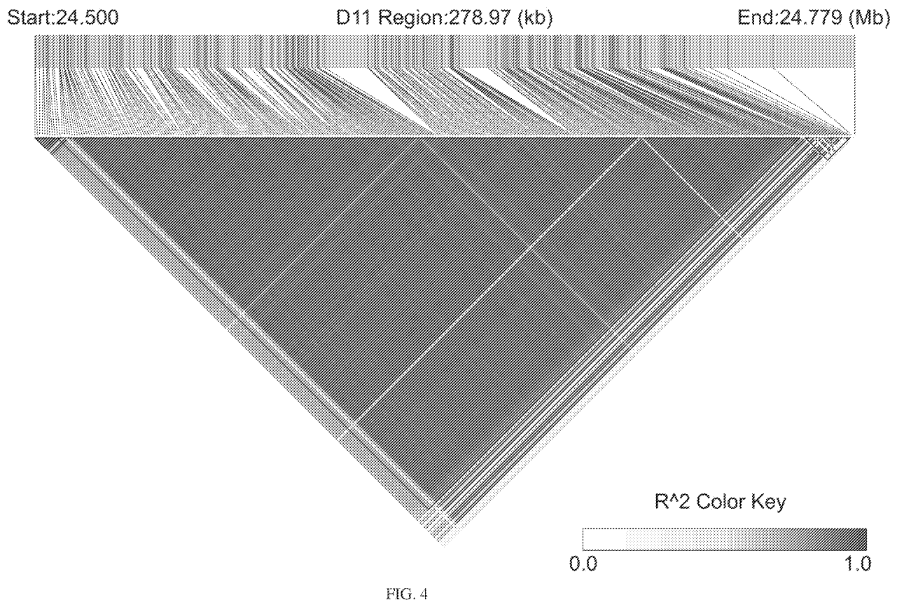

(2) The inventors' research team pre-detected three QTL intervals significantly associated with fibre length traits by genome-wide association analysis (GWAS) (He, S., Sun, G., Geng, X. et al. The genomic basis of geographic differentiation and fibre improvement in cultivated cotton. Nature Genetics, 2021,53,: 916-924.), and determined the physical locations of the three QTL intervals on chromosomes based on the linkage disequilibrium structural domains of the SNPs in the QTL intervals shown in FIG. 2, FIG. 3 and FIG. 4.

(3) According to the physical location information of SNP on chromosome, SNPs in the three QTL physical location intervals in step (2) are selected to develop KASP molecular markers. According to the physical position of SNP on chromosome, the first 30 bp sequence and the last 29 bp sequence of the physical position of SNP on chromosome are extracted from the reference genome (reference genome version of upland cotton: G.hirsutum.genome.CRI.TM-1) and combined to form a total of 60 bp sequence including SNP. The 60 bp sequence is screened, and the sequences containing missing bases, sequences containing more than 4 uncertain bases, sequences with coverage below 5X and sequences with 1-5 bases and the number of repeated sequences greater than or equal to 4 are removed. The screened 60 bp sequence is used for designing KASP molecular marker primers.

(4) The KASP molecular marker primers designed in the step (3) are further screened, 10 KASP molecular markers in each fibre length QTL physical position interval are uniformly selected, and the genotyping effect of the developed KASP molecular markers is detected by using the parent materials with known SNP. Four KASP molecular markers with good genotyping effect are screened in each physical location interval of fibre length QTL, thus obtaining the KSAP molecular markers closely linked with cotton fibre length QTL. The primer sequences and SNP information of the KASP molecular markers are shown in Table 1:

TABLE 1
Primer sequences and SNP information of KASP molecular markers

| Fibre | KASP | | | | | |
|---|---|---|---|---|---|---|
| length | marker | | | Physical | | Fibre |
| QTL | name | Primer type | Primer sequence | location | SNP | length |
| FL5 | KASP- | Allele-specific primer | gaaggtgaccaagttcatgctagta | 88,396,617 | T | Long |
| | G41 | X (SEQ ID NO. 1) | ccctgaacacatttcgatatt | bp | | |
| | | Allele-specific primer | gaaggtcggagtcaacggattagt | | C | Short |
| | | Y (SEQ ID NO. 2) | accctgaacacatttcgatatc | | | |
| | | Common primer R | tggtatcaggtattggatcggt | | | |
| | | (SEQ ID NO. 3) | | | | |
| | KASP- | Allele-specific primer | gaaggtgaccaagttcatgctggc | 88,437,416 | A | Long |
| | G42 | X (SEQ ID NO. 4) | ttgaccaattttctaaacgga | bp | | |
| | | Allele-specific primer | gaaggtcggagtcaacggattgg | | G | Short |
| | | Y (SEQ ID NO. 5) | cttgaccaattttctaaacggg | | | |
| | | Common primer R | tttgaaaaatgagagagtttgggt | | | |
| | | (SEQ ID NO. 6) | | | | |
| | KASP- | Allele-specific primer | gaaggtgaccaagttcatgctgtta | 88,459,489 | T | Long |
| | G43 | X (SEQ ID NO. 7) | taaacaatgccaccacagt | bp | | |
| | | Allele-specific primer | gaaggtcggagtcaacggattgtt | | C | Short |
| | | Y (SEQ ID NO. 8) | ataaacaatgccaccacagc | | | |
| | | Common primer R | tgggttgttcaataatgtggagc | | | |
| | | (SEQ ID NO. 9) | | | | |
| | KASP- | Allele-specific primer | gaaggtgaccaagttcatgctgcct | 88,469,638 | T | Long |
| | G44 | X (SEQ ID NO. 10) | gtaagtcatagaagaaaggt | bp | | |
| | | Allele-specific primer | gaaggtcggagtcaacggattgcc | | C | Short |
| | | Y (SEQ ID NO. 11) | tgtaagtcatagaagaaaggc | | | |
| | | Common primer R | cgtacgaactcggtgaacca | | | |
| | | (SEQ ID NO. 12) | | | | |
| FL3 | KASP- | Allele-specific primer | gaaggtgaccaagttcatgctgtg | 61,894,952 | G | Short |
| | G21 | X (SEQ ID NO. 13) | acaattttccctaaggcctc | bp | | |
| | | Allele-specific primer | gaaggtcggagtcaacggattgtg | | C | Long |
| | | Y (SEQ ID NO. 14) | acaattttccctaagg cctg | | | |
| | | Common primer R | tgcttctactccaatgccacc | | | |
| | | (SEQ ID NO. 15) | | | | |
| | KASP- | Allele-specific primer | gaaggtgaccaagttcatgcttcgt | 61,890,951 | G | Short |
| | G22 | X (SEQ ID NO. 16) | tgcgaaacatttcagag | bp | | |
| | | Allele-specific primer | gaaggtcggagtcaacggatttcg | | C | Long |
| | | Y (SEQ ID NO. 17) | ttgcgaaacattcagac | | | |
| | | Common primer R | tgtagggcaagaaaagagtgt | | | |
| | | (SEQ ID NO. 18) | | | | |
| | KASP- | Allele-specific primer | gaaggtgaccaagttcatgctttca | 61,976,868 | T | Short |
| | G23 | X (SEQ ID NO. 19) | tgtataattattacccgccct | bp | | |
| | | Allele-specific primer | gaaggtcggagtcaacggattttc | | C | Long |
| | | Y (SEQ ID NO. 20) | atgtataattattacccgcccc | | | |
| | | Common primer R | aatggtttgcgagatccttacagtc | | | |
| | | (SEQ ID NO. 21) | | | | |
| | KASP- | Allele-specific primer | gaaggtgaccaagttcatgctcgc | 62,027,231 | G | Short |
| | G24 | X (SEQ ID NO. 22) | agcactcagggatctg | bp | | |
| | | Allele-specific primer | gaaggtcggagtcaacggattcgc | | C | Long |
| | | Y (SEQ ID NO. 23) | agcactcagggatctc | | | |
| | | Common primer R | ggaccaaagcttaacgcgtg | | | |
| | | (SEQ ID NO. 24) | | | | |
| FL2 | KASP- | Allele-specific primer | gaaggtgaccaagttcatgctccat | 24,669,170 | T | Long |
| | G31 | X (SEQ ID NO. 25) | cttgttggtgtcgaatttt | bp | | |
| | | Allele-specific primer | gaaggtcggagtcaacggattcca | | G | Short |
| | | Y (SEQ ID NO. 26) | tcttgttggtgtcgaatttg | | | |
| | | Common primer R | aggacagcctaaagtcgaacg | | | |
| | | (SEQ ID NO. 27) | | | | |
| | KASP- | Allele-specific primer | gaaggtgaccaagttcatgctttgt | 24,671,829 | G | Long |
| | G32 | X (SEQ ID NO. 28) | aaggatggctatggtgg | bp | | |
| | | Allele-specific primer | gaaggtcggagtcaacggattttgt | | C | Short |
| | | Y (SEQ ID NO. 29) | aaggatggctatggtgc | | | |
| | | Common primer R | gcctgtggagccaaacaatg | | | |
| | | (SEQ ID NO. 30) | | | | |
| | KASP- | Allele-specific primer | gaaggtgaccaagttcatgctaca | 24,676,672 | T | Long |
| | G33 | X (SEQ ID NO. 31) | gagaattttcagatgctcttt | bp | | |
| | | Allele-specific primer | gaaggtcggagtcaacggattaca | | C | Short |
| | | Y (SEQ ID NO. 32) | gagaattttcagatgctcttc | | | |
| | | Common primer R | actttccagctgctgcgtat | | | |
| | | (SEQ ID NO. 33) | | | | |
| | KASP- | Allele-specific primer | gaaggtgaccaagttcatgctccct | 24,567,513 | A | Long |
| | G34 | X (SEQ ID NO. 34) | tatgcttcgtgtttctcaa | bp | | |
| | | Allele-specific primer | gaaggtcggagtcaacggattccc | | G | Short |
| | | Y (SEQ ID NO. 35) | ttatgcttcgtgtttctcag | | | |
| | | Common primer R | agactgttccttggaatcttca | | | |
| | | (SEQ ID NO. 36) | | | | |

(5) The molecular marker-assisted selection of single or multiple cotton fibre length QTLs may be realized by using the above KASP molecular markers, and the method for molecular marker-assisted selection of a single cotton fibre length QTL is as follows: using the four KSAP molecular markers in the FL5 interval in the cotton fibre length QTL screened in step (4), carrying out genotyping detection of the population materials constructed in step (1) by using the KASP molecular markers, selecting material with homozygous alleles of long fibres marked by four KASP molecules at the same time, where the selection method is to identify them by labeling fluorescent groups in primers, in which gaaggtgaccaagttcatgct corresponds to FAM fluorescence and gaaggteggagtcaacggatt corresponds to VIC fluorescence. The molecular marker-assisted selection effect of single cotton fibre length QTL is shown in Table 2:

TABLE 2
Molecular marker-assisted selection performance
of single cotton fibre length QTL

| | | QTL name | | | |
|---|---|---|---|---|---|
| | | FL5 | | | |
| Environment | | 2020 | | 2021 | |
| Genotype of | KASP-G41 | T | C | T | C |
| test samples | KASP-G42 | A | G | A | G |
| | KASP-G43 | T | C | T | C |
| | KASP-G44 | T | C | T | C |

| | | | | |
|---|---|---|---|---|
| Number of samples tested | 326 | 1216 | 361 | 1379 |
| Average fibre length (mm) | 29.57 ± | 28.21 ± | 29.73 ± | 28.91 ± |
| | 1.822 | 1.796 | 1.522 | 1.648 |

| | | |
|---|---|---|
| Difference in average fibre | 1.36 | 0.82 |
| length (mm) | | |
| t value | 12.05 | 8.56 |
| Degree of freedom | 1540 | 1738 |
| Significance | **** | **** |

The molecular marker-assisted selection method for a plurality of cotton fibre length QTLs includes the following steps: using four KSAP molecular markers in FL5 interval, four KASP molecular markers in FL3 interval and four KASP molecular markers in FL2 interval in the cotton fibre length QTLs screened in step (4), carrying out KASP molecular marker genotyping detection on the population materials constructed in step (1), selecting materials with homozygous alleles of long fibres containing 12 KASP molecular markers, where the selection method is to identify them by labeling fluorescent groups in primers, in which gaaggtgaccaagttcatgct corresponds to FAM fluorescence and gaaggteggagtcaacggatt corresponds to VIC fluorescence, and the molecular marker-assisted selection effects of multiple cotton fibre length QTLs are shown in Table 3:

TABLE 3
Molecular marker-assisted selection performance
of multiple cotton fibre length QTLs

| Environment | | 2020 | | 2021 | |
|---|---|---|---|---|---|
| Genotype of | KASP-G41 | T | C | T | C |
| test samples | KASP-G42 | A | G | A | G |
| | KASP-G43 | T | C | T | C |
| | KASP-G44 | T | C | T | C |
| | KASP-G21 | C | G | C | G |
| | KASP-G22 | C | G | C | G |
| | KASP-G23 | C | T | C | T |
| | KASP-G24 | C | G | C | G |
| | KASP-G31 | T | G | T | G |
| | KASP-G32 | G | C | G | C |
| | KASP-G33 | T | C | T | C |
| | KASP-G34 | A | G | A | G |

| Number of samples tested | 21 | 43 | 22 | 50 |
|---|---|---|---|---|
| Average fibre length (mm) | 30.33 ± | 27.23 ± | 30.51 ± | 27.71 ± |
| | 1.874 | 1.629 | 1.800 | 1.395 |
| Difference in average fibre | 3.1 | | 2.8 | |
| length (mm) | | | | |

| | | |
|---|---|---|
| t value | 6.801 | 7.172 |
| Degree of freedom | 62 | 70 |

The above-mentioned embodiments only describe the preferred mode of the present disclosure, and do not limit the scope of the present disclosure. Under the premise of not departing from the design spirit of the present disclosure, various modifications and improvements made by ordinary technicians in the field to the technical scheme of the present disclosure shall fall within the protection scope determined by the claims of the present disclosure.

What is claimed is:

1. A combination of KASP molecular markers closely linked with cotton fibre length QTL. wherein the molecular markers are KASP molecular markers KASP-G41, KASP-G42, KASP-G43 and KASP-G44 closely linked to FL5 interval in cotton fibre length QTL;
the KASP-G41 is a DNA fragment synthesized by primers with nucleotide sequences as shown in SEQ ID NO. 1, SEQ ID NO. 2 and SEQ ID NO. 3;
the KASP-G42 is a DNA fragment synthesized by primers with nucleotide sequences as shown in SEQ ID NO. 4, SEQ ID NO. 5 and SEQ ID NO. 6;
the KASP-G43 is a DNA fragment synthesized by primers with nucleotide sequences as shown in SEQ ID NO. 7. SEQ ID NO. 8 and SEQ ID NO. 9;
the KASP-G44 is a DNA fragment synthesized by primers with nucleotide sequences as shown in SEQ ID NO. 10, SEQ ID NO. 11 and SEQ ID NO. 12;
a physical location of an SNP site of the KASP-G41 is 88,396,617 bp on A07 chromosome, being a C/T mutation;
a physical location of an SNP site of the KASP-G42 is 88,437,416 bp of the A07 chromosome, being an A/G mutation;
a physical location of an SNP site of the KASP-G43 is 88,459,489 bp of the A07 chromosome, being a C/T mutation; and
a physical location of an SNP site of the KASP.G44 is 88,469,638 bp of the A07 chromosome, being a C/T mutation.

2. A KASP primer pair combination closely linked with FL5 interval in cotton fibre length QTL, wherein the KASP primer pair combination comprises primers as shown in a following table:

| S/N of SNP | KASP marker name | Primer type | S/N of sequence |
|---|---|---|---|
| SNP1 | KASP-G41 | X | SEQ ID NO. 1 |
| | | Y | SEQ ID NO. 2 |
| | | R | SEQ ID NO. 3 |
| | KASP-G42 | X | SEQ ID NO. 4 |
| SNP2 | | Y | SEQ ID NO. 5 |
| | | R | SEQ ID NO. 6 |
| | KASP-G43 | X | SEQ ID NO. 7 |
| SNP3 | | Y | SEQ ID NO. 8 |
| | | R | SEQ ID NO. 9 |
| SNP4 | KASP-G44 | X | SEQ ID NO. 10 |
| | | Y | SEQ ID NO. 11 |
| | | R | SEQ ID NO. 12. |

3. A method for identifying cotton fibre length traits by using a single cotton fibre length QTL molecular marker, comprising following steps:
S1, conducting a KASP molecular marker genotyping detection on a cotton material to be tested by using the KASP primer pair combination according to claims 2; and
S2, identifying the cotton material to be tested as a cotton plant with long fibre trait when an amplified signal as described below is read:
amplified signal of primer type X detected by the KASP-G41, the KASP-G42, the KASP-G43 and the KASP-G44 when the KASP primer pair combination is employed.

4. A kit for identifying cotton fibre length traits, comprising the primer pair combination according to claim 2.


SEQUENCE LISTING (36 sequences)

SEQ ID NO: 1 - DNA, 46 bp, synthetic construct
  source 1..46: mol_type = other DNA
gaaggtgaccaagttcatgctagtaccctgaacacatttcgatatt

SEQ ID NO: 2 - DNA, 46 bp, synthetic construct
  source 1..46: mol_type = other DNA
gaaggtcggagtcaacggattagtaccctgaacacatttcgatatc

SEQ ID NO: 3 - DNA, 22 bp, synthetic construct
  source 1..22: mol_type = other DNA
tggtatcaggtattggatcggt

SEQ ID NO: 4 - DNA, 45 bp, synthetic construct
  source 1..45: mol_type = other DNA
gaaggtgaccaagttcatgctggcttgaccaattttctaaacgga

SEQ ID NO: 5 - DNA, 45 bp, synthetic construct
  source 1..45: mol_type = other DNA
gaaggtcggagtcaacggattggcttgaccaattttctaaacggg

SEQ ID NO: 6 - DNA, 24 bp, synthetic construct
  source 1..24: mol_type = other DNA
tttgaaaaatgagagagtttgggt

SEQ ID NO: 7 - DNA, 44 bp, synthetic construct
  source 1..44: mol_type = other DNA
gaaggtgaccaagttcatgctgttataaacaatgccaccacagt

SEQ ID NO: 8 - DNA, 44 bp, synthetic construct
  source 1..44: mol_type = other DNA
gaaggtcggagtcaacggattgttataaacaatgccaccacagc

SEQ ID NO: 9 - DNA, 23 bp, synthetic construct
  source 1..23: mol_type = other DNA
tgggttgttcaataatgtggagc

SEQ ID NO: 10 - DNA, 45 bp, synthetic construct
  source 1..45: mol_type = other DNA
gaaggtgaccaagttcatgctgcctgtaagtcatagaagaaaggt

SEQ ID NO: 11 - DNA, 45 bp, synthetic construct
  source 1..45: mol_type = other DNA
gaaggtcggagtcaacggattgcctgtaagtcatagaagaaaggc

SEQ ID NO: 12 - DNA, 20 bp, synthetic construct
  source 1..20: mol_type = other DNA
cgtacgaactcggtgaacca

SEQ ID NO: 13 - DNA, 44 bp, synthetic construct
  source 1..44: mol_type = other DNA
gaaggtgaccaagttcatgctgtgacaattttccctaaggcctc

SEQ ID NO: 14 - DNA, 44 bp, synthetic construct
  source 1..44: mol_type = other DNA
gaaggtcggagtcaacggattgtgacaattttccctaaggcctg

SEQ ID NO: 15 - DNA, 21 bp, synthetic construct
  source 1..21: mol_type = other DNA
tgcttctactccaatgccacc

SEQ ID NO: 16 - DNA, 42 bp, synthetic construct
  source 1..42: mol_type = other DNA
gaaggtgaccaagttcatgcttcgttgcgaaacatttcagag

SEQ ID NO: 17 - DNA, 41 bp, synthetic construct
  source 1..41: mol_type = other DNA
gaaggtcggagtcaacggatttcgttgcgaaacattcagac

SEQ ID NO: 18 - DNA, 21 bp, synthetic construct
  source 1..21: mol_type = other DNA
tgtagggcaagaaaagagtgt

SEQ ID NO: 19 - DNA, 46 bp, synthetic construct
  source 1..46: mol_type = other DNA
gaaggtgaccaagttcatgctttcatgtataattattacccgccct

SEQ ID NO: 20 - DNA, 46 bp, synthetic construct
  source 1..46: mol_type = other DNA
gaaggtcggagtcaacggattttcatgtataattattacccgcccc

SEQ ID NO: 21 - DNA, 25 bp, synthetic construct
  source 1..25: mol_type = other DNA
aatggtttgcgagatccttacagtc

SEQ ID NO: 22 - DNA, 40 bp, synthetic construct
  source 1..40: mol_type = other DNA
gaaggtgaccaagttcatgctcgcagcactcagggatctg

SEQ ID NO: 23 - DNA, 40 bp, synthetic construct
  source 1..40: mol_type = other DNA
gaaggtcggagtcaacggattcgcagcactcagggatctc

SEQ ID NO: 24 - DNA, 20 bp, synthetic construct
  source 1..20: mol_type = other DNA
ggaccaaagcttaacgcgtg

SEQ ID NO: 25 - DNA, 44 bp, synthetic construct
  source 1..44: mol_type = other DNA
gaaggtgaccaagttcatgctccatcttgttggtgtcgaatttt

SEQ ID NO: 26 - DNA, 44 bp, synthetic construct
  source 1..44: mol_type = other DNA
gaaggtcggagtcaacggattccatcttgttggtgtcgaatttg

SEQ ID NO: 27 - DNA, 21 bp, synthetic construct
  source 1..21: mol_type = other DNA
aggacagcctaaagtcgaacg

SEQ ID NO: 28 - DNA, 42 bp, synthetic construct
  source 1..42: mol_type = other DNA
gaaggtgaccaagttcatgctttgtaaggatggctatggtgg

SEQ ID NO: 29 - DNA, 42 bp, synthetic construct
  source 1..42: mol_type = other DNA
gaaggtcggagtcaacggattttgtaaggatggctatggtgc

SEQ ID NO: 30 - DNA, 20 bp, synthetic construct
  source 1..20: mol_type = other DNA
gcctgtggagccaaacaatg

SEQ ID NO: 31 - DNA, 45 bp, synthetic construct
  source 1..45: mol_type = other DNA
gaaggtgaccaagttcatgctacagagaattttcagatgctcttt

SEQ ID NO: 32 - DNA, 45 bp, synthetic construct
  source 1..45: mol_type = other DNA
gaaggtcggagtcaacggattacagagaattttcagatgctcttc

SEQ ID NO: 33 - DNA, 20 bp, synthetic construct
  source 1..20: mol_type = other DNA
actttccagctgctgcgtat

SEQ ID NO: 34 - DNA, 44 bp, synthetic construct
  source 1..44: mol_type = other DNA
gaaggtgaccaagttcatgctcccttatgcttcgtgtttctcaa

SEQ ID NO: 35 - DNA, 44 bp, synthetic construct
  source 1..44: mol_type = other DNA
gaaggtcggagtcaacggattcccttatgcttcgtgtttctcag

SEQ ID NO: 36 - DNA, 22 bp, synthetic construct
  source 1..22: mol_type = other DNA
agactgttccttggaatcttca